(19) United States
(12) Patent Application Publication
(10) Pub. No.: US 2007/0092927 A1
(43) Pub. Date: Apr. 26, 2007

(54) Photocatalytic particles with directed and controlled redox activity

(75) Inventors: Jay M. Johnson, Dayton, OH (US); Elmo Blubaugh, Springboro, OH (US)

Correspondence Address: DINSMORE & SHOHL LLP, ONE DAYTON CENTRE, ONE SOUTH MAIN STREET, SUITE 1300, DAYTON, OH, 45402-2023, US

(73) Assignee: University of Dayton, Dayton, OH (US)

(21) Appl. No.: 11/451,139

(22) Filed: Jun. 12, 2006

Related U.S. Application Data
(60) Provisional application No. 60/689,993, filed on Jun. 13, 2005.

(51) Int. Cl. C12Q 1/26; C12M 3/00

(52) U.S. Cl. 435/25; 435/287.1; 977/902

(57) ABSTRACT

A modified particle. The modified particle comprises: a semiconductor particle which is photoactivatable; and a modifier molecule attached to the semiconductor particle, wherein the modifier molecule includes an electrochemically reversible redox active site which is photoexcitable. Bioconjugated nanoparticle probes, Redox-NSs and methods of using these modified particles are also described.

### CROSS-REFERENCE TO RELATED APPLICATIONS

This application claims the benefit of provisional application Ser. No. 60/689,993 entitled PHOTOACTIVATED NANOPARTICLES WITH DIRECTED AND CONTROLLED REDOX ACTIVITY filed Jun. 13, 2005.

### BACKGROUND OF THE INVENTION

This invention relates generally to particles, and more particularly to particles which have been modified to provide directed and controlled redox activity.

Photoactivation of semiconductors, including various metal oxides and fullerenes, is a well studied process and involves promotion of valence band electrons to the conduction band upon absorption of photons with energy greater than the bandgap. Upon photoactivation of particulate semiconductors, electron-hole pairs are created at the particle surface which can be exploited for various processes at the solvent/surface interface. Various metal-oxide semiconductor particles including SnO₂, WO₃, ZnO, TiO₂, PbO, V₂O₅, Bi₂O₃, Fe₂O₃, CdO, Cu₂O, and CuO exhibit a large oxidation potential on their surface due to photogenerated holes. This phenomenon has been used extensively to break down organic pollutants photocatalytically to less troublesome inorganic species.

Unmodified TiO₂ in particular has been used extensively in environmental remediation because it is non-toxic, inexpensive, and has a high band gap potential which results in the ability to oxidize many organic pollutants. It has also been used to construct photoelectrochemical cells consisting of dye sensitized nano-porous TiO₂ films. These cells are based on charge transfer that occurs upon illumination, between the sensitizer attached to the TiO₂ surface and the TiO₂. A reducing agent is present in the electrolyte to regenerate the reduced form of the sensitizer and mediate electron transport to the counter electrode to complete the circuit.

In general, the non-selectivity of the process and the rapid recombination of the photogenerated electron-hole pairs are the major problems limiting the utility of photocatalysis using semiconductor particles or nanoparticles. It is believed that interfacial electron transfer on unmodified TiO₂ occurs via surface Ti (IV) atoms which, because they are co-ordinated with solvent molecules, trap conduction band electrons. Hole transfer is thought to involve surface oxygen. Previous studies have demonstrated that surface modification/functionalization of semiconductor nanoparticles allows charge pairs to be further separated across the nanoparticle/modifier interface upon photoactivation. This can slow down or limit electron-hole recombination and, in some cases, provide some improvement in selectivity. However, to our knowledge, there has been no report of photoactivated or photoexcited nanoparticle catalysts that were oxidation/reduction (redox) “tuned” via surface modification of the nanoparticles with electrochemically reversible redox sites such that the modified nanoparticles (MNPs) could be used to carry out more specific photocatalytic transformations at their surface in complex reaction matrices.

U.S. Pat. Nos. 6,271,130 and 6,410,935 describe the use of films of TiO₂ nanoparticles that are modified with organic ligands such that specific metal ions can be localized/conjugated to the surface and subsequently photoreduced in order to create metal deposits (Cu, Ag, Au) on the surface of the nanoparticle. This process can be used to nanopattern the surface of the film with conductive metal traces for building nanoelectronic circuits and components. However, any/all of these metals would be reduced onto the nanoparticle surface if they were present and the appropriate ligands were attached to the surface. In other words, the specificity of the process is associated with the selection of the ligand and the fact that only metal ions of the desired metal are present when photoactivation occurs. Thus, the matrix is relatively simple and controlled, and highly specific redox transformations are not required. No mention is made of the concept of fine tuning the redox properties of these liganded metal sites to carry out controlled and directed chemical transformations.

Inorganic nanoparticles and modified nanoparticles (MNPs) have potential in diagnostic and therapeutic applications. Fluorescent dye labeled proteins and reporter genes have been used extensively as probes and sensors to study cell signaling, in vivo. However, the use of nanoparticles or MNPs as intracellular nanosensors or nanobiosensors has a number of potential advantages to these more classical techniques. Most importantly, they can provide increased detectability, due to the fact that multiple copies of fluorescent dye molecules can be added to the nanoparticle surface, and multifunctionality since multiple sensing or localization elements can be attached to the same particle. Bioconjugated nanoparticle probes (BNPs) are MNPs that have been modified with molecules with a specific biological activity and have been used for in vivo optical imaging and for drug delivery. Many of these BNPs are based on quantum dot (QD) cores, which are generally composed of CdSe, and could have some innate toxicity (Cd is not an FDA-approved injectable substance). Gold bioconjugated nanoparticles (Au-BNPs) have been used extensively for in vitro scanning, electron microscopy, and immunodiagnostics, and as well as in some in vivo applications.

TiO₂ nanoparticles have been conjugated with single strand DNA to create a multifunctional “bioconjugate” nanoprobe (BNP) that retained both the biological activity of the attached DNA and the intrinsic photocatalytic activity of the TiO₂ nanoparticle. Conjugation to the TiO₂ surface was accomplished using dopa, dopamine or other diphenols and unwanted side reactions averted via further functionalization with glycidyl isopropyl ether. Photoactivation of the TiO₂ with UV light caused these BNPs to have photo-induced endonuclease activity. Gel electrophoresis and PCR were used to verify that illumination of hybridized particles resulted in the cleavage of nucleotide bonds and release of DNA from the BNP at random, short distances (up to 50 base pairs) from the TiO₂ core. It was proposed that this process probably proceeds via both charge migration and multi-step electron “hopping” mechanisms through the nucleotide backbone of the DNA and, ultimately, results in cleavage of the DNA at the point(s) where electropositive holes accumulate.

This approach may have specific applications related to, for example, gene therapy. However, it does not incorporate the means for breaking specific chemical bonds. It does not utilize a specifically engineered redox site or sites (“molecular wire”) to direct and control the electrical potential applied to the DNA. Charge flow may be directed down the DNA backbone, but the electrical potential applied is relatively high and uncontrolled. Thus, the DNA is blown apart at random distances from the nanoparticle core.

In U.S. Pat. No. 6,677,606, Rajh et. al. describe a method for selective binding of and detecting target molecules that are bound to selective affinity molecules which are attached through modifiers to the surface of TiO₂ or other metal oxide nanoparticles. The modifier molecules used to couple the affinity molecules to the nanoparticle surface include dopa, dopamine, and other bidentate ligands (these are not electrochemically reversible). Upon binding of the affinity molecules with their respective target molecules, changes in the charge pair separation characteristics at the nanoparticle modifier interface, upon photoactivation, are effected and detected via Electroparamagnetic Resonance (EPR) or absorbance spectroscopy. The changes in the spectral signatures can thus be used to detect and quantify the target molecules. However, this invention is specifically directed to molecular detection, and no mention is made of using modifiers that include an electrochemically reversible redox site and/or the attendant benefits.

There are a number of colorimetric or fluorescent redox active dyes available that have been used for intracellular redox studies. However, none of these are electrochemically reversible. Accordingly, they provide only a snapshot or a cumulative estimate of the redox status and/or verify the presence of specific redox species. They do not allow the real time and dynamic observation of the intracellular redox status.

Electrochemical cells have been described which have been used to provide oxidizing or reducing equivalents to enzymes for biosynthetic industrial processes. However, these are relatively macro in size, are complicated, and require physical contact between the enzyme and the electrode for electron transfer to occur. These systems are too large and complicated to be inserted into biological cells. In addition, colorimetric and fluorescent dyes are available that have been used with varying success to determine the intracellular concentrations of some metabolites. However, these are not generally reversible and/or applicable to use in live cells. Regardless, they cannot be used to monitor on-demand and dynamically the intracellular concentrations of important analytes like lactate and glucose. None of these technologies allows the real time and dynamic measurement of important, intracellular metabolites.

Therefore, there is a need for modified particles that incorporate further means for enhanced and prolonged charge separation such that more selective and effective redox modifications can be achieved using photoactivation or photoexcitation.

### SUMMARY OF THE INVENTION

The present invention meets this need by providing a modified particle. The modified particle comprises: a semiconductor particle which is photoactivatable; and a modifier molecule attached to the semiconductor particle, wherein the modifier molecule includes an electrochemically reversible redox active site which is photo excitable.

Another aspect of the invention is a bioconjugated nanoparticle probe. The bioconjugated nanoparticle probe comprises: a semiconductor nanoparticle which is photoactivatable; a modifier molecule attached to the semiconductor particle, wherein the modifier molecule includes an electrochemically reversible redox active site which is photoexcitable; and a bioactive molecule attached to the modifier molecule.

Another aspect of the invention involves a method of reversibly inhibiting enzyme activity. The method comprises: providing a modified particle comprising: a semiconductor particle which is photoactivatable; a modifier molecule attached to the semiconductor particle, wherein the modifier molecule includes an electrochemically reversible redox active site which is photoexcitable and an enzyme inhibitor; and photoactivating, photoexciting, or both the modified particle so that charge flow is directed in a controlled manner from the core of the modified particle to the exterior surface of the modified particle and wherein the electrochemically reversible redox active site is oxidized or reduced and the exterior surface of the metal oxide particle or a species at the solution/semiconductor particle surface interface is reduced or oxidized. The oxidized or reduced electrochemically reversible redox active site in the modifier molecule subsequently oxidizes or reduces the enzyme inhibitor attached to the modifier molecule, rendering the enzyme inhibitor non-inhibiting.

Another aspect of the invention involves a method of monitoring intracellular redox status. The method includes providing a redox nanosensor comprising a modified nanoparticle comprising: a semiconductor nanoparticle which is photoactivatable; a modifier molecule attached to the semiconductor nanoparticle, wherein the modifier molecule includes an electrochemically reversible redox active site which is photoexcitable; photoactivating, photoexciting, or both the modified nanoparticle so that charge flow is directed in a controlled manner from the core of the modified nanoparticle to the exterior surface of the modified nanoparticle and wherein the electrochemically reversible redox active site is oxidized or reduced and the exterior surface of the semiconductor particle or a species at the solution/semiconductor particle surface interface is reduced or oxidized; and monitoring the fluorescence of the redox nanosensor.

Another aspect of the invention involves a method of catalyzing an enzyme reaction. The method includes providing a modified nanoparticle comprising: a semiconductor nanoparticle which is photoactivatable; a modifier molecule attached to the semiconductor nanoparticle, wherein the modifier molecule includes an electrochemically reversible redox active site which is photoexcitable; and an oxidase enzyme attached to the modifier molecule; photoactivating, photoexciting, or both the modified nanoparticle so that charge flow is directed in a controlled manner from the core of the modified particle to the exterior surface of the modified nanoparticle and wherein the electrochemically reversible redox active site is oxidized or reduced and the exterior surface of the semiconductor particle or a species at the solution/semiconductor particle surface interface is reduced or oxidized. The oxidized or reduced redox active site in the modifier molecule subsequently oxidizes or reduces the enzyme attached to the modifier molecule in order to catalyze or activate the oxidase enzyme reaction.

### BRIEF DESCRIPTION OF THE DRAWINGS

FIG. 1 shows a schematic concept of one embodiment of a titanium dioxide-based bioconjugated nanoparticle probe.

FIG. 2 shows a cyclic voltammogram of the 5,5′ Dicarboxylate-Bipyridine, Ruthenium Bis Bipyridine Complex-Quinazoline Src Kinase inhibitor.

FIG. 3 shows a cyclic voltammogram of the 5,5′ Dicarboxylate-Bipyridine, Ruthenium Bis Bipyridine Complex.

FIG. 4 shows the fluorescence spectra obtained at 430 nm excitation on the free 5,5′ Dicarboxylate-Bipyridine, Ruthenium Bis Bipyridine Complex.

FIG. 5 shows the fluorescence spectra obtained at 370 nm excitation for bare P25 TiO₂ nanoparticles, free 5,5′ Dicarboxylate-Bipyridine, Ruthenium Bis Bipyridine Complex, and P25 TiO₂ nanoparticles modified with adsorbed 5,5′ Dicarboxylate-Bipyridine, Ruthenium Bis Bipyridine Complex.

FIG. 6 shows the fluorescence spectra obtained at 405 nm excitation for bare P25 TiO₂ nanoparticles, free 5,5′ Dicarboxylate-Bipyridine, Ruthenium Bis Bipyridine Complex, and P25 TiO₂ nanoparticles modified with adsorbed 5,5′ Dicarboxylate-Bipyridine, Ruthenium Bis Bipyridine Complex.

FIG. 7 shows the difference emission spectra obtained at 430 nm excitation on TiO₂ nanoparticles modified with the 5,5′ Ru metal complex minus that obtained on the bare TiO₂ nanoparticles.

FIG. 8 shows emission spectra of TiO₂ nanoparticles modified with the 5,5′ Ru complex at an excitation wavelength of 370 nm taken over one hour during intermittent illumination at 430 nm.

FIG. 9 shows the results of an assay that demonstrates that the TiO₂-inhibitor BNP first inhibits and then after photoactivation/photoexcitation releases Src kinase activity, in vitro.

FIG. 10 shows the results of an assay that demonstrates that the TiO₂-inhibitor BNPs do not affect cell membrane permeability of C18-4 cells.

FIG. 11 shows the results of an assay that demonstrates that the TiO₂-inhibitor BNPs do not affect the mitochondrial activity of C18-4 cells.

FIG. 12 shows the results of functional assays in which the relative number of viable C18-4 cells was measured using the MTS assay after treatment with TiO₂-inhibitor BNPs.

FIG. 13 shows the fluorescence intensity decay (excitation at 488 nm; emission at 519 nm) of TiO₂-inhibitor BNPs in C18-4 stem cells over time under constant illumination at 405 nm.

FIG. 14 shows a fluorescence spectrum (excitation at 488 nm) of the TiO₂ nanoparticles modified with the 5,5′ Ru complex.

FIG. 15 shows a schematic concept of one embodiment of a titanium dioxide-based redox nanosensor.

FIG. 16 shows a schematic of one method of metal complex (redox amino acid) formation.

FIG. 17 shows a schematic of the general structure of one embodiment of the metal complexes and their attachment to the TiO₂ particle.

FIG. 18 shows one embodiment of an enzyme based nanocatalyst and the expected photocatalytic oxidation of glucose.

### DESCRIPTION OF THE INVENTION

This invention relates to the design of modified semiconductor particles and nanoparticles that can be photoactivated and/or photoexcited. Previous studies have demonstrated that surface modification/functionalization of semiconductor nanoparticles allows charge pairs to be separated across the nanoparticle/modifier interface upon photoactivation. The current invention relates to the exact nature of these surface modifiers. More specifically, it relates to the design and use of modified nanoparticles (MNPs) in which the attached modifier includes redox active sites. These redox sites (single or multiple) are electrochemically reversible and can mediate electron transfer. Suitable redox sites are believed to be, but are not limited to, a series of polypyridyl metal complexes based on ruthenium, osmium, or rhenium. Similar complexes based on these metals have been described in the literature and are generally electrochemically reversible and photoexcitable. Upon photoexcitation they form a metal-to-ligand charge transfer (MLCT) state in which the metal center is oxidized and the ligand is reduced. This MLTC state can then decay by both non-radiative and radiative pathways. In many cases, depending upon a number of criteria, the relative rates of these processes are such that the extent of radiative decay in the form of fluorescence is significant. Thus, many of these metal complexes are fluorescent in the reduced state. Their fluorescence is generally long lived, and is therefore amenable to time filtered detection which can improve detection limits especially in complex biological media. Also, by using different ligand substitutions, the redox potential (E_{1/2}) or redox energetics of the complex can be varied over a relatively large range (0.1-1.0 V vs. SCE).

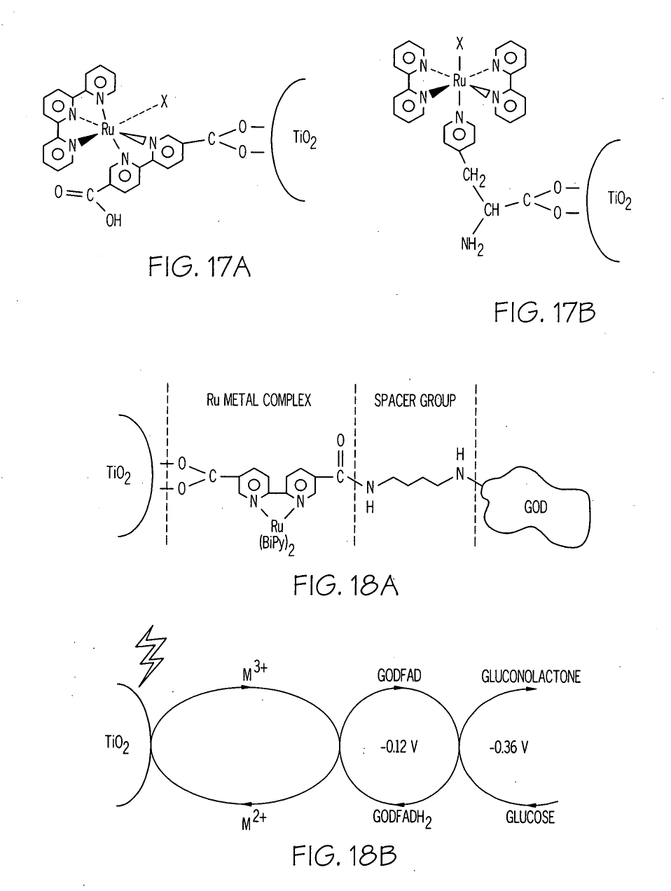

It should be noted that the attachment of these redox sites can be done either directly or indirectly. Direct attachment involves adsorption through chemical functionality (—COOH, —OH, —C═O groups, or groups containing sulfur or phosphorous atoms) present on the redox site or metal complex (for example, —COOH groups on the dicarboxylate bypyridine ligand (see FIG. 17) to co-ordinatively unsaturated Ti (IV) surface sites. Indirect attachment involves first reacting the redox site or metal complex with bifunctional spacer groups containing —COOH, —OH, —C═O groups, or groups containing sulfur or phosphorous atoms, including, but not limited to, spacers groups containing dopa; dopamine; and modified or unmodified diphenols, hydroxylphenyls, siloxanes, thiols, thiol esters, thiol carbamides, and phosphonates, which are then adsorbed to the TiO₂ through the interaction of the free —COOH, —OH, —C═O groups, or the groups containing sulfur or phosphate in the spacer to co-ordinatively unsaturated Ti(IV) sites.

These reversible redox sites effectively behave as molecular wires and allow the charge generated (holes or electrons) at the surface of the nanoparticle to be trapped and utilized more effectively than if the nanoparticle were unmodified or modified with non-reversible electrochemically active groups. Upon photoactivation (UV light) of the TiO₂ nanoparticle, they effectively direct charge (holes or electrons) flow in a controlled manner from the nanoparticle core toward the exterior surface of the MNP. In some cases, charge flow could be initiated via photoexcitation (usually with visible light) of the metal complex attached to the TiO₂. In this case, charge separation is initiated at the metal complex redox site, and charge (electrons or holes) flows toward the TiO₂. In either case, the metal redox site is oxidized (usually) or reduced and a reduction (usually) or oxidation reaction, respectively, occurs at the surface or solution/surface interface of the TiO₂ particle. The judicious choice of the specific, reversible redox active sites would allow the control of both the thermodynamics and kinetics of subsequent electron transfer reactions occurring between the redox active sites and other chemical functionality, either within the backbone of the modifier (intramolecular) or with other near-by chemical species (intermolecular).

Further enhancements to the basic invention include: 1) the incorporation of reversible redox active sites that are photoexcitable in the oxidized and/or reduced state 2) the incorporation of redox active sites that are photoexcitable in the oxidized and/or reduced state and are fluorescent such that the spatial location of the modified nanoparticles could be determined in real-time by following this fluorescence and 3) conjugation of the modifier containing the reversible redox active site with bioactive molecules to form a photoactivated and/or photoexcited BNP. In the case where the reversible redox sites are fluorescent in only one of the redox forms or where the two redox forms fluoresce at different wavelengths, the fluorescence could be monitored in order to monitor the progression of the desired redox transformation.

Further bioconjugation of the core TiO₂ particle could allow this novel BNP to have multiple functionalities, including: 1) enhanced transport across membranes of biological cells; 2) localization within the cell; 3) sensing or indication of biochemical events; and 4) on-demand influence of biochemical pathways through photoactivation/photoexcitation in real-time. Not all functionalities need be present for the BNP to be effective. Furthermore, and importantly, use of fluorescent redox sites on the modifier would allow the visualization and localization of the BNP within the cell.

The use of nanoparticles and MNPs as opposed to micro- or larger particles (modified and unmodified) is a benefit in a number of applications where the increased surface area to volume ratio is important. However, the smaller nanosized particles can be more difficult to use and retrieve than larger particles. Nanoparticles are defined as particles having a diameter of from 1 to 1000 nanometers. For intracellular applications or studies, nanosized particles or MNPs with diameters from 1 to 50 nm are preferred because they more readily pass through the cell membrane, probably via passive diffusion. Regardless, the present invention is not limited to the use of nanoparticles but could find applications involving larger particles sizes as well.

The potential applications of this invention are many because photoactivation/photoexcitation could essentially be used to perform specific chemical (redox) reactions, in-situ and on-demand without a physical contact/interface. Approaches using modified or unmodified semiconductor nanoparticles have been demonstrated, but they do not have the requisite specificity and, in general, are useful only in situations where broad specificity is desirable or tolerable.

The major benefits of the current invention include, but are not limited to, further stabilization of and prolonged charge separation at the modifier/semiconductor interface. This is especially important in cases where the intent is to perform more selective redox reactions (inter- or intramolecular) at the surface of the nano-particle. Some of the advantages of this invention come from the ability to engineer modifiers attached to nanoparticle conjugates such that the resulting photocatalysts can be used to perform specific chemical transformations, in-situ, without undesirable side reactions. This is an advantage especially when the reaction matrix is relatively complex.

The present invention has tremendous potential in the development of photoactivated/photoexcited BNPs for use in in-vivo studies on biological cells where specificity is an important consideration. These studies would both utilize these BNPs and lead to the development of other potential applications of photoactivated/photoexcited BNPs including biosensors, therapeutics, diagnostics and gene therapy.

One example is directed to solving a problem involving cell signaling pathways. Signaling pathways in mammalian cells drive an extraordinary array of biological processes that are important in maintaining healthy tissue and, if disrupted, can lead to diseases such as cancer. The ability to probe and influence these pathways will have great value in drug discovery and development.

One use of the photoactivated/photoexcited BNP involves the study of signaling pathways in spermatogonial stem cells that involve an enzyme called Src kinase. There is great interest in the spermatogonial stem cells because of their importance for understanding basic mechanisms of stem cell renewal versus differentiation, for the treatment of infertility, for the development of contraceptives, and for the understanding of the etiology of testicular cancer. Src kinase is involved in this signaling pathway and has been implicated in the control of cell division, cell survival, and cell motility in other systems. However, how Src works and what it does is still not fully understood. Presently, approaches to understanding Src function include microinjection of interfering Src mutant proteins, microinjection of inhibiting antibodies, transfection of plasmid vectors harboring dominant negative or positive Src genes, retroviral gene transfection and, lately, pharmacological inhibitors. Except for the use of pharmacological inhibitors, all these techniques are invasive and might promote changes in the cellular environment unrelated to Src function. Also, many of the techniques are time consuming, require a relatively large amount of purified protein to assess the outcome, and do not provide a real-time assessment of Src activity or cellular location. The use of photoactivated/photoexcited BNPs would greatly facilitate the real-time study of signaling pathways in small populations of cells, such as stem cells.

In vivo assays are a complement to in vitro assays, since the ultimate goal of studying signal transduction pathways is to evaluate cell behavior (proliferation, differentiation, motility, etc.) in normal situations and diseases. For all the reasons cited above, novel real-time and non-invasive technologies are needed to unravel the molecular mechanisms involved in cellular signaling pathways. In vivo assays using non-toxic BNPs, which are readily able to penetrate the plasma membrane, would meet these requirements.

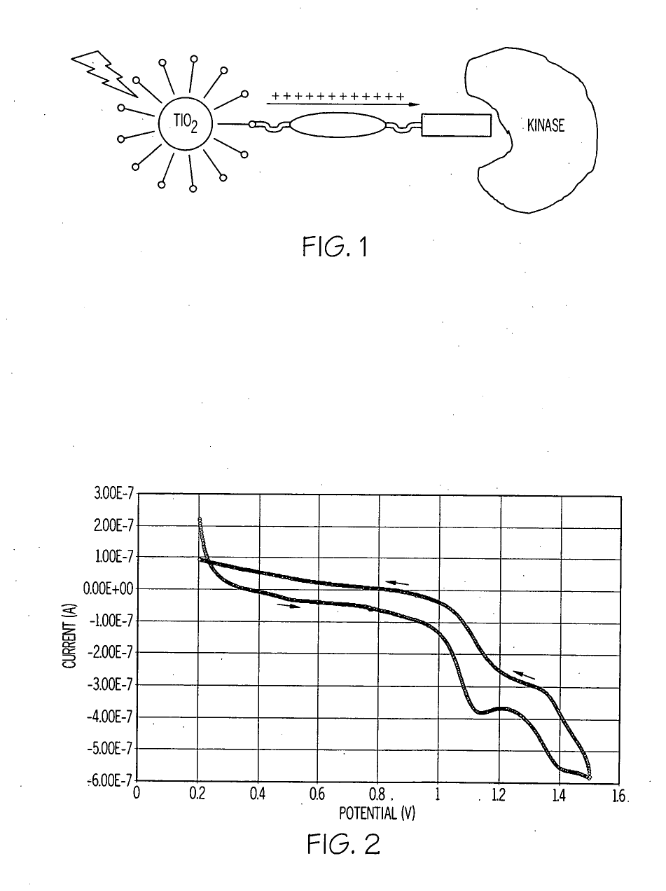

One embodiment of the subject invention involves a BNP based on titanium dioxide nanoparticles which are molecularly “wired” to an Src kinase inhibitor through a Ruthenium(II)-polypyridyl-metal complex/spacer conjugate. (FIG. 1 and structure below.) The goal with the TiO₂-inhibitor BNP is to inhibit Src kinase activity until a certain point, at which time the TiO₂-inhibitor BNP is photoactivated (or photoexcited), the oxidizing charge travels down the Ru²⁺ containing modifier to the inhibitor, and the inhibitor is oxidized and inactivated. This allows Src kinase activity and all of the downstream signaling pathways to proceed.

Development of TiO₂-BNPs

Core Nanoparticle

Titanium dioxide nanoparticles (P25) were purchased from Sigma-Aldrich, Inc. These nanoparticles are reported to have a diameter of 40 nm or less and a surface area larger than 20 m²/gram. Ultrasonication of the nanoparticle/water (or 1 mM HCl solution) mixture was used to redissolve the titanium dioxide nanoparticles and form a colloidal solution. Formation of the colloidal solution was accomplished using a 130 watt ultrasonicator at a power setting of 40% for six minutes, with an on/off dwell time of 2/1. These dispersions of titanium dioxide were stable for greater than 24 hours.

As shown schematically in FIG. 1 (not drawn to scale), TiO₂ nanoparticles or beads (2-50 nm diameter) are modified with a molecular wire (20-50 Å, shaded oval) and connected to a kinase inhibitor (box). UV or visible light (lightning bolt) photoactivates the TiO₂ or photoexcites the metal complex site in the molecular wire which, in either case, generates an oxidizing positive charge that is conducted down the molecular wire to inactivate the inhibitor and allow the signaling cascade to proceed. In the process, a reducing charge (electron) travels to the TiO₂, and a reduction reaction occurs at the surface of the TiO₂. Alternatively, visible light could be used to photoexcite the metal complex redox site, which would also result in the oxidative charge traveling to the inhibitor and reducing charge to the TiO₂ as before.

Fmoc-Based Solid State Synthesis of Redox modifier—Src Inhibitor Conjugate.

The redox modifier-Src inhibitor conjugate was a 5,5′Bipyridine-putrescene-amino benzoic acid Dimethoxy-quinazoline. It was synthesized using Fmoc-based solid state synthesis on a Wang resin. The first step was the coupling of the 5,5′-Dicarboxylate-Bipyridine to the Wang resin. The second step involved the coupling of Fmoc-Putrescene to the free carboxylic group presented by the Bipyridine. The free amino group from the Fmoc-Putrescene was attached to the free carboxylic group of the bipyridine. The Fmoc protecting group was removed, and the resulting free amino group was coupled to Fmoc-amino benzoic acid. A further Fmoc deprotection step was performed on the amino group of the benzoic acid, with subsequent attachment of the 1-Chloro-6,7-Dimethoxy quinazoline molecule. This sequence of steps completed the synthesis of the complexing agent for the Ruthenium (II) Bis-Bipyridine Bipyridine Inhibitor molecule.

The next step involved the reaction of Ruthenium (II) Bis(Bipyridine) cis-Dichloro with the Bipyridine-containing inhibitor ligand. The completed Ruthenium (II) metal complex/Src Kinase Inhibitor molecule was cleaved from the Wang resin with trifluoroacetic acid/dichloromethane. The metal complex-inhibitor was then recrystallized from acetonitrile/diethyl ether. Proton NMR showed confirmation of the molecular structure (Table I). Preliminary UV-Vis analysis indicated that the main spectral absorbance peak was centered around 437 nm with a shoulder peak at 501 nm.

TABLE I
Proton NMR peak shifts of the metal complex inhibitor

| Chemical Group | Shift Observed | |
|---|---|---|
| methylene —CH2— | 1.5 to 2.0 | ppm |
| Amide —NH— | 2.5 to 3.75 | ppm |
| Methoxy H | 4.0 | ppm |
| Quinazoline ring H | 6.5 to 9.0 | ppm |

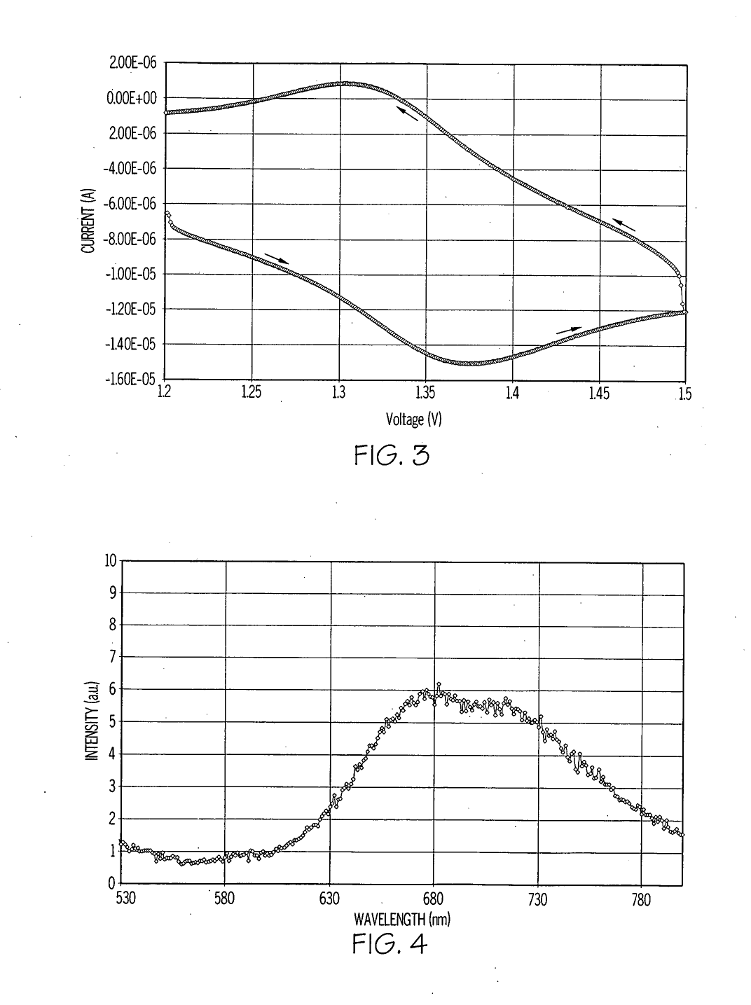

The electrochemical behavior of the conjugate in acetonitrile, with 0.25 M tetraethyl ammonium hexafluorophosphate (TEAPF₆) added as an electrolyte, at a platinum working electrode was also determined (FIG. 2). A cyclic voltammogram was performed by measuring current flow as a function of applied potential at a scan rate of mV/sec in a 0.05 mM solution of the conjugate. Oxidation peaks with potentials of about 1.150 volts and 1.400 volts versus the Ag/AgCl reference were observed. There was a return reduction peak located at 1.350 volts, but no further reduction activity was noted from 1.300 to 0.00 volts. These peaks were assigned to two redox couples. First, the chemically irreversible oxidation at 1.150 volts is due to the direct electrochemical oxidation of the quinazoline inhibitor. Second, the oxidation/reduction peaks for the +1.400 and +1.350-volt peaks are assigned to the Ru (III)/Ru (II) redox couple. The accuracy of the latter peak assignment was confirmed by the cyclic voltametric data obtained on the free, 5,5′ Dicarboxylate-Bipyridine, Ruthenium Bis Bipyridine Complex (FIG. 3). In this case, the free complex (2 mM) was dissolved in acetotonitrile/0.25 M TEAPF₆ and a glassy carbon electrode was used. The cyclic voltammogram was recorded at a scan rate of 100 mV/sec. An oxidation peak was observed at about +1.380 volts with a reversible reduction at about +1.320 volts. The differences in the observed peak potentials (free complex vs. conjugated) are not considered to be significant and could be related to the fact that the redox behavior of the complex is slightly affected by conjugation to the spacer and inhibitor portion of the molecule. These data taken together confirm that the Ruthenium metal complex is electrochemically reversible and could act as a mediator for the oxidation of the quinazoline inhibitor.

Coupling of the Redox Modifier-Src Inhibitor Conjugate to the TiO₂ Nanoparticle

The ruthenium(II)/quinazoline inhibitor molecule was then successfully attached by adsorption to the surface of TiO₂ nanoparticles as determined by visual and spectroscopic evaluation, as well as subsequent biochemical testing. Although not wishing to be bound by theory, this attachment is believed to involve the interaction of the free —COOH group of the Ruthenium complex to the co-ordinatively unsaturated Ti⁴⁺ sites on the TiO₂. The structure of the TiO₂ based inhibitor BNP directed to Src kinase (TiO₂-inhibitor BNP) is depicted below. This structure was confirmed by FTIR and NMR. 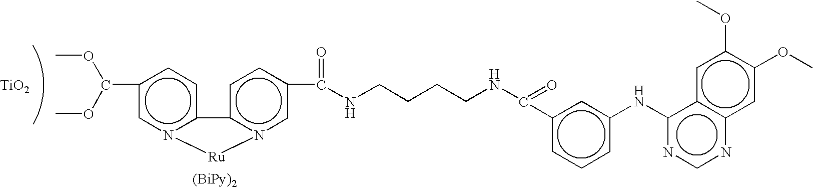
Fluorescence Spectroscopic Evaluation of the TiO₂ Modified with the 5,5′ Dicarboxylate-Bipyridine, Ruthenium Bis Bipyridine Complex (5,5′ Ru complex).

These studies were carried out in order to better elucidate the charge transfer interactions and the related photodynamics between the 5,5′ Ru complex and the TiO₂ particle. In this case, the putrescine spacer and inhibitor portion of the conjugate were not present. Fluorescence spectra were measured for the bare TiO₂ nanoparticles, free 5,5′ Ru complex, and TiO₂ nanoparticles modified with the 5,5′ Ru complex using various excitation wavelengths from 300 to 430 nm. The free 5,5′ Ru metal complex is weakly fluorescent (emission centered at about 700 nm) when excited at wavelengths from about 300-340 nm, which is known to be associated with ligand charge transfer interactions.

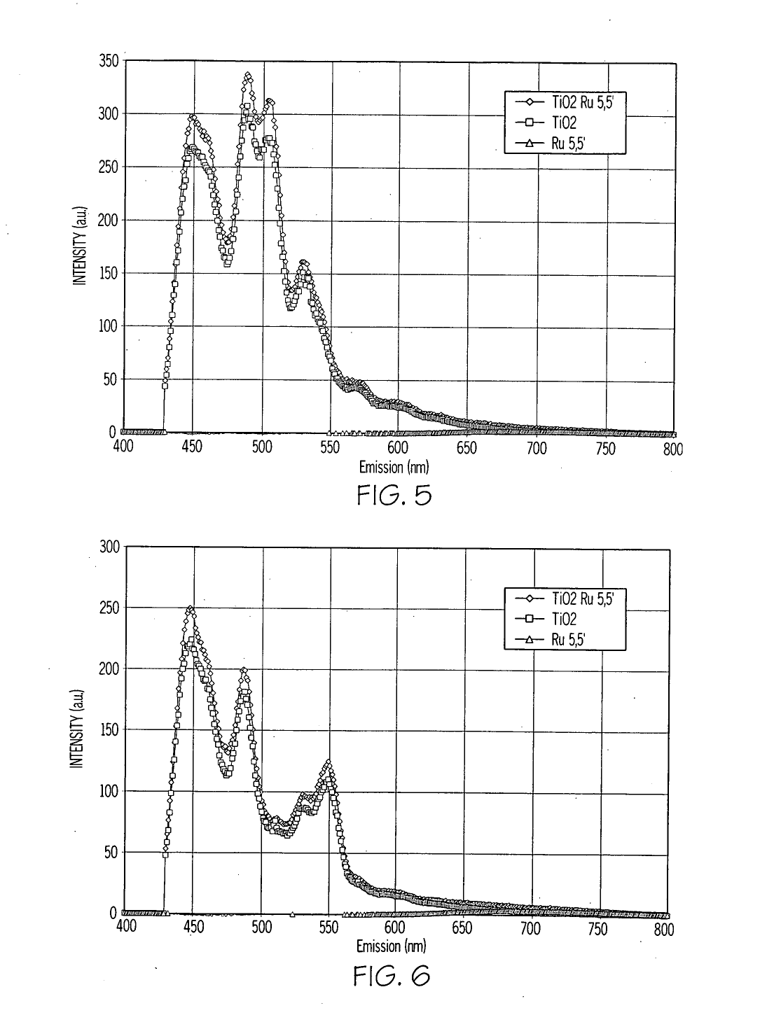

The absorbance and emission spectra for this 5,5′Ru complex have been reported. The absorbance spectrum shows a relatively broad and weak absorbance peak from about 400 to 490 nm. The emission spectra, at excitation wavelengths of 290 nm and 430-450 nm, have as many as three very weak intensity emission peaks, including one at around 700 nm. However, two of these emission peaks were thought to be due to either impurities or instrumental artifacts. Emission spectra on the highly purified 5,5′ Ru complex displayed only one weak emission peak at about 700 nm regardless of the excitation wavelength from 300 to 450 nm. FIG. 4 illustrates a typical emission spectra obtained on the free 5,5′ Ru complex at an excitation wavelength of 430 nm. FIGS. 5 and 6 illustrate representative fluorescence spectra obtained at excitation wavelengths of 370 and 405 nm, respectively, on bare P25 TiO₂ nanoparticles, free 5,5′ Dicarboxylate-Bipyridine, Ruthenium Bis Bipyridine Complex and P25 TiO₂ nanoparticles modified with adsorbed 5,5′ Dicarboxylate-Bipyridine, Ruthenium Bis Bipyridine Complex. The 5,5′ Ru complex was adsorbed to the TiO₂ as described above, and all spectra were recorded at equimolar equivalent concentrations. All measurements were done in a Cary Eclipse Fluorescence spectrometer.

These fluorescence spectra indicate that the fluorescence behavior of the free complex and the bare TiO₂ nanoparticles are not significantly different when they are free vs. when they are attached to each other. In addition, it was observed that for both bare TiO₂ and TiO₂ with the 5,5′ Ru complex attached, the TiO₂ does not fluoresce significantly when illuminated at wavelengths between 300 and 365 nm (data not shown). However, when illuminated at from 370 to 430 nm, which is below the TiO₂ bandgap energy, there are multiple emission peaks centered at from 450 to 550 nm (see FIGS. 5 and 6). These TiO₂ peaks also trend downward in intensity as the excitation wavelength is increased from 370 to 430 nm. Although not wishing to be bound by theory, we believe that the explanation for this behavior at excitation wavelengths at and below 365 nm is that the energy is sufficient to promote electrons in the TiO₂ valence band to the conduction band. Further the emission peaks associated with the TiO₂ that are present at excitation wavelengths above 365 nm are believed to be due to radiative decay of photoactivated surface sites on the TiO₂ nanoparticle. The fluorescence emission at about 700 nm from the adsorbed 5,5′ Ru complex trends downward from 300 to 365 nm and then begins to increase again above about 405 nm excitation (not shown). This fluorescence emission is so weak that it is obscured by the TiO₂ emissions at 370 and 405 nm excitation wavelengths, as shown in FIGS. 5 and 6.

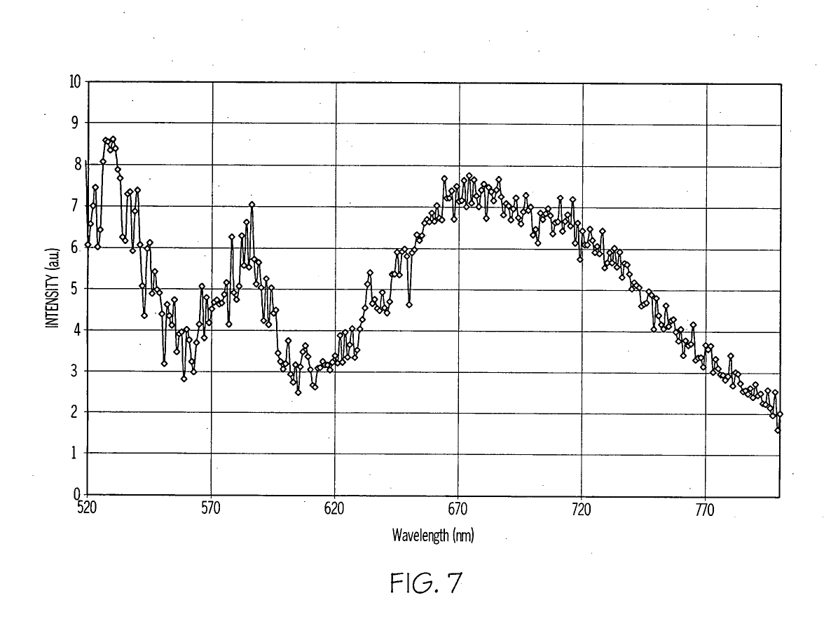

FIG. 7 is a difference spectra at an excitation wavelength of 430 nm in which the emission spectra for the bare TiO₂ is subtracted from that of the TiO₂ with adsorbed 5,5′ Ru metal complex. Although the spectrum is quite noisy below 600 nm, it clearly shows that the 5,5′ Ru complex emits at 700 nm (compare FIG. 7 to FIG. 4) even when the complex is attached to the TiO₂ surface. These results suggest that it might be possible to effectively photoexcite the Ru metal complex attached to the TiO₂ surface at excitation wavelengths with energies less than the band gap of TiO₂ (above about 365 nm wavelength) and achieve charge separation originating at the Ru atom. This should result in oxidation of Ru⁺² to Ru⁺³, electrons being pumped across the 5,5′ Ru complex/TiO₂ interface, and reduction of redox active species at the TiO₂ surface. This process would be expected to be cumulative and proceed more rapidly at greater fluences as might be achieved with a laser.

FIG. 8 illustrates a series of fluorescence spectra obtained at an excitation wavelength of 370 nm over one hour time with intermittent illumination at 430 nm. The spectral scans with excitation at 370 nm required about 30 seconds to perform. Between spectral scans the sample was continuously illuminated at 430 nm. Periods of illumination at 430 nm were 9 min. 30 sec., and total illumination time at 430 nm was about one hour. It is apparent that under these conditions, the fluorescence peaks between 450 and 550 nm associated with the TiO₂ decay relatively rapidly. In fact, it was observed that the fluorescence continues to decay if the illumination time is extended to several hours (not shown). This phenomenon was not observed either with bare TiO₂ or with TiO₂ modified with other less effective Ru metal complexes (data not shown). These peaks are believed to be associated with surface sites on the TiO₂ that are being photocatalytically reduced and rendered non-fluorescent under illumination at 430 nm. Since this process is occurring at a wavelength (430 nm) well above the band gap of the TiO₂, it is believed that the decay of these fluorescence peaks is a direct indication that photocatalytic charge separation, subsequent oxidation of the 5,5′ Ru complex and reduction of or at the TiO₂ surface is being accomplished.

It is expected that it should be possible to effect charge separation at the 5,5′ Ru metal complex—TiO₂ interface via photoactivation of the TiO₂ since it a very energetic process. However, because the process described above is occurring at light energies below those required for photoactivation of the TiO₂, it must be occurring through the photoexcitation of the Ru atom in the 5,5′ Ru complex. These results suggest that the requisite charge separation and desired oxidation of the Ru atom can be accomplished through either photoactivation of the TiO₂ or photoexcitation of the attached 5,5′ Ru complex. This is significant because the latter process can be accomplished at longer wavelengths or under less energetic conditions than the former process. Therefore, it should be more amenable for selectively performing the desired photocatalytic process in environments where there may be reactive chemical and/or sensitive biological species, such as are found in the intracellular matrix of biological cells.

In Vitro Assay of the TiO₂ Based Inhibitor BNP Directed to Src Kinase.

The TiO₂ BNP with redox modifier and pharmacological Src inhibitor attached (TiO₂-inhibitor BNP) was assayed to determine whether it would inhibit Src kinase in vitro and whether that inhibition could be reversed by photoactivating/photoexciting the TiO₂-inhibitor BNP. The TiO₂-inhibitor BNP was assayed at increasing concentrations against 3 units of purified Src kinase using a protein tyrosine kinase assay kit (Chemicon). The assay kit is based on binding of a phosphotyrosine monoclonal antibody to a substrate that has been phosphorylated by Src kinase. This monoclonal antibody is conjugated to horseradish peroxidase, and is visualized by a colorimetric reaction. Determination of Src kinase inhibition and photoactivated-release of that inhibition was accomplished by preparing two identical sample sets in triplicate with varying concentrations of nanoprobes. One sample set was then exposed to UV light for 10 minutes, and one was not exposed to UV light. These sample sets were then assayed for phosphorylation of the Src kinase substrate as described above.

Src kinase activity in samples that have not been exposed to UV light, and in samples that were incubated with TiO₂-inhibitor BNPs and then exposed to UV light (λ=365 nm) for 10 minutes was compared to autophosphorylation by Src kinase (positive control) and general inhibition of Src kinase by EDTA (negative control). (n=3 for all samples.)

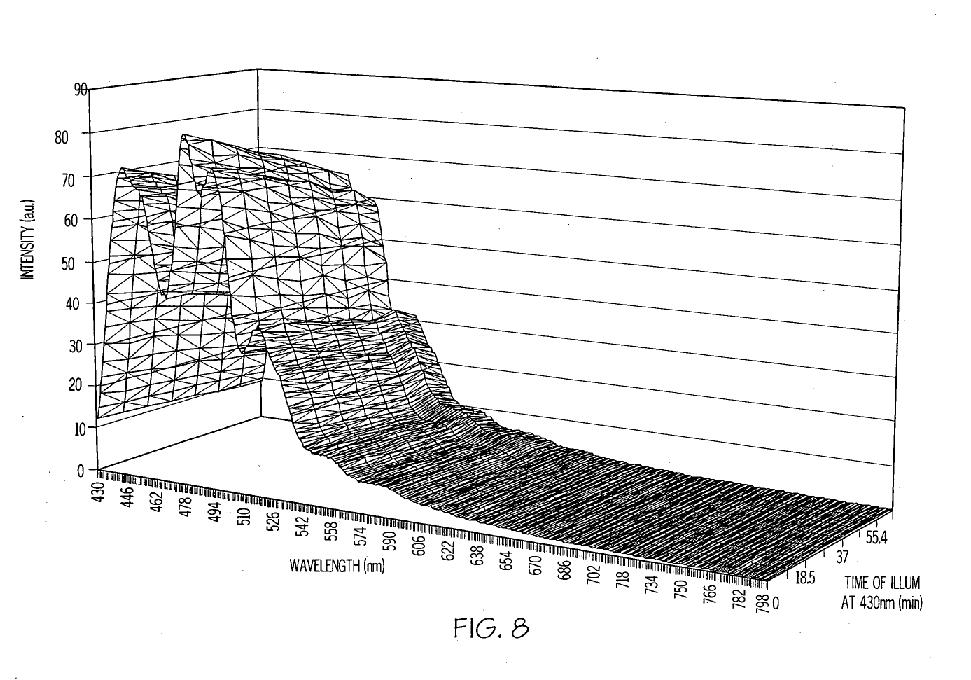

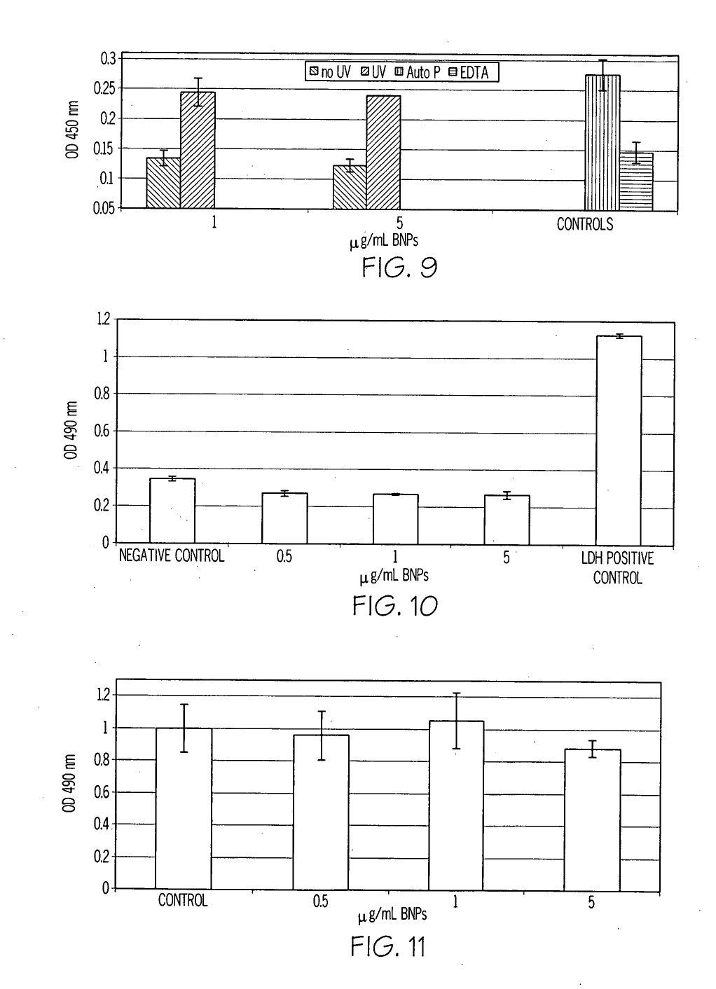

As shown in FIG. 9, the TiO₂-inhibitor BNP inhibited Src kinase activity in vitro at BNP concentrations of 1 and 5 μg/mL in samples that had not been exposed to UV light. In this assay, the absorbance measured at 450 nm (Y-axis) is directly proportional to Src kinase activity. The level of inhibition observed with the TiO₂-inhibitor BNP present was slightly lower than that of the general inhibitor EDTA, which was used as a negative control. After a 10 minute exposure to long wavelength UV light (λ=365 nm), this inhibition was reversed, and the Src kinase was active. The level of recovered activity was similar to that of the positive control sample (FIG. 9), indicating that there was nearly a complete reversal of inhibition. These data indicate that this TiO₂-inhibitor BNP works as designed: the probe inhibits Src kinase activity until photoexcitation/photoactivation, which causes an oxidizing charge to travel down the Ru-metal complex wire and oxidize the inhibitor so it is no longer effective. Thus, we have demonstrated that the nanoprobe can photoreversibly inhibit enzyme activity.

Cell Viability and Cytotoxicity Evaluations of TiO₂-Inhibitor BNPs

BNPs developed for intracellular studies should have minimal or no detectable cytotoxicity at the concentrations of intended use. Accordingly, the cytotoxicity of the TiO₂-inhibitor BNP was assessed in a spermatogonial stem cell line (C18-4) using a number of different assays to assess cell viability in the presence of the TiO₂-inhibitor BNP.

LDH Assay

The spermatogonial stem cell line, C18-4, was incubated with the TiO₂-inhibitor BNPs for 48 hrs, after which the lactate dehydrogenase (LDH) assay was performed to determine whether these BNPs disrupt membrane integrity and thereby decrease cell viability. This assay was performed on cells that had been incubated with BNPs for 48 hours. FIG. 10 illustrates these results. In this plot, the “OD at 490” (Y-axis) is indirectly proportional to membrane integrity. Cells that had not been incubated with BNPs were used for the negative control. The positive LDH control is also shown. As indicated in FIG. 10, the TiO₂-inhibitor BNPs did not affect membrane integrity at 0.5, 1, and 5 μg/mL nanoprobes, compared to the control. These data demonstrate that, at concentrations at which this BNP effectively inhibits and releases Src kinase activity (see FIG. 9), the BNPs are not toxic to the cells by this measure.

MTS Assay

The mitochondrial activity of C18-4 stem cells incubated for 48 hrs with TiO₂-inhibitor BNPs was determined using a mitochondrial activity (MTS) assay. These results are illustrated in FIG. 11. In this plot, the “OD at 490” (Y-axis) is directly proportional to mitochondrial activity. These data indicate, that in the presence of BNPs at concentrations of 0.5, 1 and 5 μg/mL, the mitochondrial activity was unaffected compared to the control. Cells that had not been incubated with BNPs were used for the control. Again, these results indicate that the TiO₂-inhibitor BNP is not affecting normal cell viability at concentrations that are effective to confer the activity of the BNP.

Apoptosis Assay

C18-4 cells were grown to 80% confluency in 6-well culture plates, and incubated with TiO₂-inhibitor BNPs for 24 hours at a concentration of 1 μg/ml. Then half of the cultures were photoactivated with UV light (λ=365 nm), and all samples further cultivated for another day. Control cultures without BNPs were used as well. Cultures were washed three times in cold Phosphate Buffered Saline (PBS). 1 microliter of Component A (YO-PRO-1) and 1 microliter of Component B (propidium iodide) of the Vybrant Apoptosis Assay Kit (Invitrogen) were added to each culture. Cells were incubated for 25 minutes on ice, then analyzed by fluorescence microscopy. A minimum of 500 cells were counted. Live cells show only background fluorescence, apoptotic cells show only green fluorescence, and necrotic cells show both red and green fluorescence.

The results of this study showed that there were no significant differences between sample cultures with BNPs in terms of either the % cells in apoptosis (less than 3%) and the % of viable cells (>96%). This result was the same regardless of whether or not sample cultures containing BNPs were exposed to UV light. Furthermore, the same results were obtained on control cultures which did not contain BNPs both with and without UV exposure. The conclusion is that the Src-inhibitor nanoparticles with or without photoactivation/photoexcitation do not promote apoptosis or negatively effect the viability (non-apoptotic and non-necrotic cells) of C18-4 cells.

Reactive Oxygen Species (ROS) Assay

Aminophenyl fluorescein (APF) (Invitrogen-Molecular Probes) is a ROS indicator with specificity for hydroxyl radical, peroxynitrite anion and hypochlorite anion. C18-4 cells were cultured until 80% confluency in 6-well culture plates, and incubated with TiO₂-inhibitor BNPs for 24 hours at a concentration of 1 μg/ml. Half of the cultures were photoactivated/photoexcited with UV light (λ=365 nm) for 10 min, and all samples further cultivated for 1 hour, 24 hours, and 48 hours before the ROS assay was performed. Control cultures without BNPs were used as well. For the ROS assay, the cells were incubated with a final concentration of 5 μM of APF for 1 hour at 37° C., then washed with fresh medium and fluorescence observed at 515 nm.

The results of this study indicated that there were no significant differences after UV exposure and further cultivation for one hour between the cultures that were not UV activated and those that were UV activated. The results also suggested that approximately 1.5% of the cells produce ROS in culture without the BNPs and that about the same number of cells produce ROS when TiO₂-inhibitor BNPs are present. At 24 hours cultivation after UV exposure, the results were similar with no significant differences between UV and no UV exposure and controls without BNPs. Similar results were obtained after 48 hours cultivation after UV exposure. However, there is an increase in the overall ROS production by the C18-4 cells over time. This increase is not due to the presence of the BNPs, nor the consequence of UV photoactivation; it is probably due to nutrient depletion as the cultures age and become confluent.

Taken together, the results described above indicate that the TiO₂-inhibitor BNPs are effective and non-toxic as measured by a number of different assays at low concentrations (up to 1 μg/ml) in the cells.

In Vivo Studies

Functional Assay: Relative Number of C18-4 Cells after Treatment with TiO₂-Inhibitor BNPs

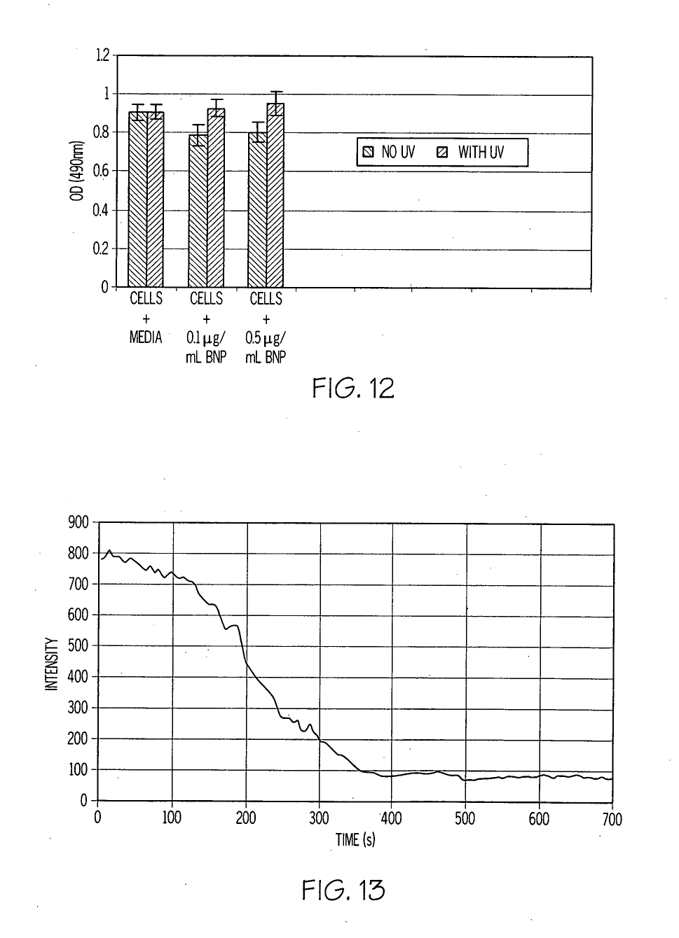

Studies were performed to demonstrate that the TiO₂-inhibitor BNPs inhibit and release Src kinase activity intracellularly, as designed. Since Src kinase is known to promote cell proliferation, this should be observable by measuring the rate of cell proliferation in the absence and presence of the TiO₂-inhibitor BNPs and with and without photoexcitation/photoactivation. C18-4 cells were seeded in 96-well microtiter plates at a concentration of 10,000 cells/well. TiO₂-inhibitor BNPs were added after 24 hours, when the cells started the log phase. Two days later, half of the cultures were irradiated with UV light at λ=365 nm. After 48 hours, a mitochondrial activity (MTS) assay was performed to measure the relative cell number (mitochondrial activity detected is proportional to the number of viable cells). Cells that had not been incubated with BNPs were used for the control (Cells+Media). Results are provided in FIG. 12. These results show that the TiO₂-inhibitor BNPs inhibited the proliferation of the C18-4 cells at a concentration of 0.1 and 0.5 μg/ml by about 10-15%. After photoactivation/photoexcitation, the number of C18-4 cells returned to the original level. This indicates that the release of the inhibition after photoactivation/photoexcitation also occurs in vivo. Although the inhibitory effect is small (10-15%), the results are statistically significant for concentrations of 0.1 and 0.5 μg/ml nanoparticles. The small observed effect could be related to the presence of alternative pathways for cell proliferation that are activated when Src kinase is inhibited. In this case, lower concentrations of TiO₂-inhibitor BNPs were used than in the in-vitro assay (FIG. 9) because it was observed by Transmission Electron Microscopy (TEM) that the BNPs were significantly concentrated within cells.

Observation of the Fluorescence Decay Associated with the TiO2-Inhibitor BNP in C18-4 Cells Under Constant Laser Illumination.

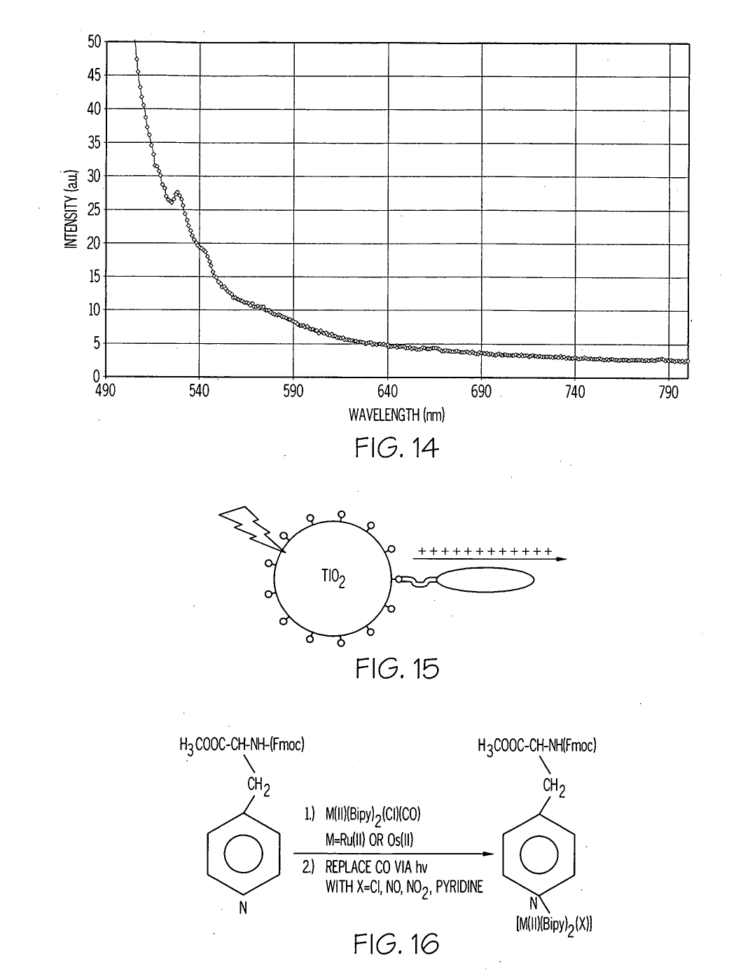

Cells were imaged with an Olympus Confocal microscope (FV-1000) equipped for live cell imaging. This system is a high resolution spectral imaging system that can be used to image and photoexcite or photoactivate, simultaneously, the TiO₂-inhibitor BNPs within live cells. Wavelengths which can be used to image or photoexcite/photoactivate are limited by available laser lines. However, because the laser light is extremely intense (about 500 to 1000 times greater compared to the source used in the fluorescence spectrometer work described above) and focused, it should be possible to photoexcite or photoactivate much more rapidly and at less than optimal wavelengths. In this case, we used a 405 nm laser line to photoexcite the 5,5′ Ru complex on the TiO₂-inhibitor BNP and a 488 nm laser line to excite the surface states on the TiO₂ nanoparticle. The fluorescence of these TiO₂ surface sites was monitored at 519 mm. C18-4 cells were incubated with TiO₂-inhibitor BNPs (0.5 μg/ml) for 24 hrs. Cells were then washed to remove any BNPs that did not enter the cells and were incubated for an additional 24 hrs to let the cells recover before they were imaged. A circular area of about 200 sq. microns, which appeared to contain just one C18-4 cell, was photoexcited and imaged, simultaneously. Within this area, the fluorescence intensity of the TiO₂ surface sites (519 nm, emission; 488 nm, excitation) was monitored while 405 nm laser light was used to photoexcite the TiO₂-inhibitor BNP. The fluorescence intensity at 519 nm is plotted as a function of time and displayed in FIG. 13 as the same 200 sq. micron area is photoexcited at 405 nm. There is little change in the fluorescence intensity during the first 150 seconds, followed by a rapid decrease in the intensity between 150 to 450 seconds. After 450 seconds, the fluorescence intensity is relatively low but constant. These results for the TiO₂-inhibitor BNP are similar to those described above in the fluorescence spectroscopy studies and depicted in FIG. 8 for the TiO₂ modified with the 5,5′ Ru complex but without the Src inhibitor portion of the BNP. However, in this case the 405 nm laser line was used to photoexcite the Ru atom in the TiO₂-inhibitor BNP, instead of 430 nm light of much lower intensity from the fluorescence spectrometer. Also, in this case, the fluorescence of the TiO₂ surface sites was monitored by exciting these sites with the 488 nm line and measuring the emission at 519 nm while simultaneously photoexciting the Ru atom at 405 nm, as opposed to excitation at 370 nm (after illumination at 430 nm for 9 minutes 30 seconds) and scanning the entire emission wavelength range. Because the fluence from the laser is so much greater than that of the spectrometer source, the decay of the fluorescence observed with laser photoexcitation is much more rapid (a few minutes vs. several hours). To further confirm that the TiO₂ surface sites could be imaged by exciting at 488 nm and monitoring fluorescence at 519 nm, the fluorescence spectra in the fluorescence spectrometer of the TiO₂ nanoparticles modified with the 5,5′ Ru complex with excitation at 488 nm were recorded. These results are shown in FIG. 14 and indeed show that there is a small intensity emission peak located at around 520 nm that is probably associated with the TiO₂ surface sites. As mentioned before, the intensity of this emission peak would be expected to be much greater (500 to 1000×) using laser excitation vs. the xenon lamp source in the fluorescence spectrometer.

The observed behavior is consistent with the interpretation that photoexcitation (intracellular) of the Ru atom within the TiO₂-inhibitor BNP can be used to effect charge separation at the 5,5′ Ru complex/TiO₂ interface, oxidation of the Ru atom from Ru⁺² to Ru⁺³, and reduction of or at the surface TiO₂ sites. Further, the in-vitro and in-vivo assay results taken with these results further support the conclusion that the TiO₂-inhibitor BNP behaves intracellularly within C18-4 cells as designed, in that, after oxidation of the Ru atom from Ru⁺² to Ru⁺³, it in turn, oxidizes the Src inhibitor portion of the TiO₂-inhibitor BNP and removes the inhibitory effect on Src kinase enzyme.

Other TiO₂-inhibitor BNPs that would function similarly and provide photocatalytically reversed enzyme inhibition can be prepared. The appropriate specific or non-specific enzyme inhibitor(s) would be attached to the terminal end of the TiO₂-redox modifier MNP in a manner similar to that described above for the TiO₂-inhibitor BNP directed to Src kinase.

The TiO₂-inhibitor BNP described above without the conjugated Src inhibitor is within the general scope of the invention. In other words, the TiO₂-redox modifier construct where the modifier contains reversible redox active groups (for example, ruthenium) is the core of the concept/invention. The addition of the Src inhibitor (or another enzyme inhibitor) to the redox modifier to form the conjugate which is ultimately attached to the TiO₂ to form the TiO₂-inhibitor BNP is an enhancement on the concept.

Redox Nanosensors

In another embodiment, the invention can be used as a Redox Nanosensor. The TiO₂ nanoparticles modified with reversible redox sites can be used as Redox Nanosensors (Redox-NSs) to monitor the redox status of the microenvironment, for example, within biological cells. In this case, the kinetics and energetics associated with the intermolecular interaction of the Redox NS with intracellular redox components is determined by the choice of the specific redox sites. Further, the use of redox sites that are fluorescent in only one redox form allows the Redox particles to be tracked and their redox status to be monitored optically.

The potential applications of this embodiment mostly involve cellular assays in which the intracellular redox status is monitored in order to study basic cellar processes or determine the effects of toxins or potential drug candidates. Such cellular assays could be used for the high throughput screening of drug candidates and for cell based biosensors for toxin detection.

Existing colorimetric and fluorescent redox dyes have been used for limited purposes to assess the intracellular redox status. However, these are not electrochemically reversible, and therefore do not allow the dynamic redox status of the intracellular environment to be monitored in real time. Other benefits associated with using a MNP based Redox NS as opposed to molecular sensing species or dyes include the ability to concentrate the molecular detection species on the surface and to further modify the MNP surface to provide additional functionality.

It is known that surface modification/functionalization of semiconductor nanoparticles (e.g., TiO₂, VO₂, or WO₂) allows, upon photoactivation, charge pairs to be separated across the nanoparticle/modifier interface. The modifiers used here are reversible redox active sites, and the MNP is a Redox Nanosensor (Redox-NS). The judicious choice of the specific redox active sites would allow the control of both the thermodynamics and kinetics of subsequent electron transfer reactions occurring between the redox active sites on the Redox-NS and redox active species present in the intracellular environment. Additionally, these redox sites can be fluorescent in one of the redox forms but not the other. This allows the visualization and localization of the Redox-NS within the cell. More importantly, it allows the redox potential or status of the redox site to be determined by monitoring the fluorescence intensity of the Redox-NS, in real-time, and because the redox modifier sites are electrochemically reversible, dynamically. Also, because these sites can interact with intracellular redox species, the redox status of the redox sites on this “Redox Nanosensor” reflects the redox status of the local intracellular environment. The redox sites or modifiers are typically based on complexes of ruthenium, osmium or rhenium metal which are generally fluorescent in the reduced form but not the oxidized form. When the core TiO₂ particle is photoactivated, charge separation occurs across the TiO₂-redox modifier interface, and the redox modifiers can be either oxidized or reduced depending on the redox properties of the modifier and the nature of its attachment to the TiO₂ particle. Alternatively, this process could be initiated via photoexcitation of the redox modifier. Thus, by monitoring the fluorescence of the Redox-NS during and/or after photoactivation/photoexcitation, the Redox-NS can be calibrated, and the re-equilibration of the Redox-NS with redox species in the local cellular environment can be observed. This should allow further elucidation of the exact nature of redox species that are present in the intracellular environment and their respective concentrations.

Further bioconjugation or modification of the Redox-NS with a Cell Localization Signal (CLS) Peptide could allow the Redox-NS to be transported to specific cell compartments, for example, the nucleus and the redox status within and across specific cell compartments determined in real time under various conditions.

The interactions between the various redox active species present in the intracellular environment are extensive and very complex, and the resulting redox balance depends on a number of factors. These include the concentrations of specific redox active species and the energetics (E⁰′, formal potential, or E_{1/2}, half-wave potential) and kinetics associated with their respective interactions involving homogeneous electron exchange. Obviously, a delicate redox balance should be maintained such that ROS levels are not allowed to exceed toxic levels and yet normal cellular functions can be maintained. Furthermore, the optimal redox balance and the resulting electrochemical (redox) activity or potential is likely to be different in different sub-cellular compartments. If ROS levels exceed the antioxidant capacity of the cell or sub-cellular compartment, then they become toxic and lead to oxidative injuries or stress. Oxidative stress is observed in various disease states including cancer, acquired immunodeficiency syndrome (AIDS), Alzheimer's disease, rheumatoid arthritis, and Parkinson's, or when cells are exposed to various toxins or drugs. Presently, there is no way to observe and study directly the imbalance in the intracellular redox environment that results in oxidative stress, and which is caused by, and symptomatic of, the disease. The use of redox nanosensors (Redox-NSs) based on modified nanoparticles (MNPs) to observe real-time changes in the intracellular redox or electrochemical potential or activity in diseased and normal cells both in the absence and presence of various drugs and drug candidates would allow further elucidation of disease mechanisms and effects and improved evaluations of drugs for efficacy and safety.

Extracellular potentiometric measurements have been used to study intracellular redox activity in various cell types including CHO, CH27 and L929 cells. This was accomplished using a light activated potentiometric sensor (LAPS) and ferri-/ferrocyanide as a mediator to measure extracellular potential. Menadione/menadiol was used as a “carrier” mediator to shuttle electrons across the cell membrane and to couple the intracellular redox activity with ferri-/ferrocyanide which in turn coupled with the LAPS. This study demonstrated that both equilibrium (redox potential) and non-equilibrium (redox activity) measurements of redox status can be made depending on which mediators are present and cellular conditions. However, because this approach uses soluble mediators and electrodes which are extracellular, it can not be used to monitor the intracellular redox status in real time.

Photoactivated/photoexcited TiO₂ based Redox-NSs functionalized with reversible, redox active sites could accomplish this. The nature of these redox active groups and further modifications to the TiO₂ surface will depend on the intracellular compartment and redox species being targeted. Commercially available, as well as in-house prepared nanoparticles synthesized according to Paunesku (Nat. Matls., Vol. 2, 2003, p. 343-346) could be used.

Two approaches could be used for synthesizing the redox active metal complexes/sites. In either case, the metal complex will be based on either ruthenium or osmium (or possibly rhenium), and after construction will be attached to the nanoparticle through the binding of co-ordinatively unsaturated Ti⁴⁺ sites on the surface with carboxylate oxygen atoms (FIG. 17).

These Redox-NSs will be fluorescent-based sensors for measuring the redox status (potential and/or activity) of the local cellular environment under various conditions. As shown schematically in FIG. 15, TiO₂ nanoparticles (15-20 nm diameter) are modified with redox complexes/sites (shaded oval) containing either an osmium or ruthenium complex which is attached to the TiO₂ nanoparticle through carboxylate or hydroxyl groups and the co-ordinatively unsaturated Ti⁴⁺ sites (small shaded dots). UV or visible light (lightning bolt) will photoactivate the TiO₂ or photoexcite the metal complex site which generates an oxidizing positive charge that flows from the center of the particle out and in-turn oxidizes the attached metal complexes (redox sites).

These particles should behave in a manner similar to a chemically modified potentiometric indicator electrode, except that fluorescence will be monitored instead of potential. Before photoactivation, the ratio of oxidized to reduced sites on the Redox-NS will be adjusted until equilibrium is reached, consistent with the local solution potential. The measured fluorescence will be proportional to the number of reduced sites and indicative of the local equilibrium solution potential, as defined by the Nernst equation. In the case where the redox conditions are not at equilibrium, the redox activity will be determined under controlled conditions and changes monitored in response to various test conditions. In either case, upon photoactivation or photoexcitation, the TiO₂ will oxidize all the attached redox sites and fluorescence, due to the reduced redox sites, will disappear. The time course of the reappearance of fluorescence and the final equilibrium (in some cases) fluorescence will be measured and will be dependent on diffusion, the kinetics of interaction with and the redox potentials(E_{1/2}) of soluble redox active molecules present in the local cellular environment and the E_{1/2} of the redox complexes on the Redox-NS.

Synthesis of the Metal Complexes/Redox Sites.

Among the criteria for selection of an appropriate redox couple are the half wave potential (E_{1/2}), and that either the reduced or oxidized form should be fluorescent. The redox potential (E_{1/2}) of the complex should be as close as possible to the intracellular redox or solution potential (ISP), or otherwise selected to optimize the interaction with targeted, intracellular redox active species. The intracellular solution potential is expected to depend on the cellular compartment and other conditions, and is expected to be in the +200 to +600 mV vs. SCE range. The osmium and ruthenium complexes to be synthesized are expected to be fluorescent in the reduced form (excitation at about 460 nm and emission at about 650 nm for Ru(II) and about 690 nm excitation and about 775 nm emission for the Os(II) complexes), but not in the oxidized form.

One method of making suitable compounds will involve the synthesis of a library of amino acids containing either specific osmium or ruthenium complexes which are redox active. The novel, ruthenium (osmium) (II)-polypyridyl containing amino acids can be made from a synthetic amino acid, Fmoc-L-4Pyridyl alanine-OMe, (Synthetech Inc.) Analogous rhenium complexes might also be fabricated. The panel in FIG. 16 gives a general synthetic route for metal complex-amino acids, where the ligand field is substituted with two different ligands. The starting amino acid-ligand metal complex can be synthesized from the reaction of the Fmoc/methyl ester protected 4PyAla amino acid to give the mono-substituting ligand displayed in FIG. 16. This ligand will be reacted with the metal bipyridine, monochloro, monocarbonyl complex (M(Bipy)₂,Cl CO), as indicated in the first step above the arrow. The second step in the synthesis involves substitution of the carbonyl via a second ligand, X, which can be any of the following: Cl⁻, NO, NO₂, Pyridine. By substituting these ligands for the carbonyl group, a total of eight metal complexes containing either ruthenium (4) or osmium (4) can be synthesized which have E_{1/2} values that span the range of +1.30 to +0.200 volts vs. SCE. This entire process can be performed on a solid phase (Wang resin) after first attaching the Fmoc-L-4Pyridyl alanine-OMe via the methyl ester group to the resin.

Another approach will involve the solution synthesis of a similar set of mono-substituted terpyridyl/bipyridyl-dicarboxylate metal complexes based on ruthenium or osmium. Analogous rhenium complexes might also be fabricated. The trichloro-terpyridyl metal complex is prepared first and is then reacted with dicarboxylate bypyridine to form the mono-substituted (Cl⁻) terpyridyl/bipyridyl-dicarboxylate metal complex. The Cl⁻ group in the mono-substituted complex will be thermally replaced with NO, NO₂, or pyridine. A total of eight similar metal complexes, with the identical substitutions as in the first approach and containing either ruthenium (4) or osmium (4) can be synthesized.

The complexes synthesized using the two different approaches but with the same substitutions are expected to have similar E_{1/2} values and each set is expected to span the range of +1.30 to +0.200 volts vs. SCE. Regardless of which synthetic approach is selected these metal complexes/redox sites will be attached in a similar manner through carboxyl groups present on the ligands to the TiO₂. The general structure of the complexes formed using the two different synthetic approaches and their attachment to the TiO₂ surface is shown in FIG. 17. The ruthenium metal complexes are shown but the osmium (or rhenium) complexes will be analogous. X can be Cl⁻, NO₂, NO, or pyridine. FIG. 17 A shows the terpyridyl/bipyridyl dicarboxylate metal complex, and FIG. 17 B shows the synthetic redox amino acid containing the bipyridyl metal complex. In FIG. 17, the terpyridyl/bipyridyl dicarboxylate complex (A) is shown attached to the TiO₂ nanoparticle through only one of the available —COOH groups. Actually, because the approach is simpler, fabrication of Redox-NSs could be done using both available —COOH groups on the dicarboxylate bipyridyl ligand for attachment.

This embodiment of the invention is expected to have a number of uses. In particular, it will be applicable in any situation in which there is a need to monitor the redox status in a microenvironment both dynamically and in real time. These would include applications involving intracellular measurements, as well as applications involving other microenvironments. The measurement of the intracellular redox status across various compartments would have applicability for cellular based assays for high throughput screening and drug discovery and toxin detection.

There is currently no sensor or method available for measuring intracellular redox status dynamically and in real time. Further, there are no reports of nanoparticle based redox sensors. The Redox-NS design also allows for enhanced sensitivity and detectability compared to molecular approaches since redox sites are concentrated on the nanoparticle surface. The nanoparticle design also allows the incorporation of other modifiers on the surface to provide additional functionality. Other advantages of the design associated with the ability to photoactivate TiO₂ or photoexcite the redox site and the fact that only one redox form of the Redox-NS is fluorescent include the ability to calibrate the Redox-NS, in-situ, and to dynamically monitor the re-equilibration of the Redox-NS with redox species present in the intracellular environment.

Photocatalytic Enzyme Based Nanocatalysts and Nanobiosensors

Another aspect of the invention involves the construction of a bioconjugated nanoprobe (BNP) based on a TiO₂ nanoparticle that is first modified with electrochemically reversible redox sites (redox modifier) which are photoexcitable. In this case, an oxidase enzyme (for example, glucose oxidase) is attached to the core TiO₂ particle through the redox modifier such that when the redox site is photoexcited, charge separation occurs at the redox site which results in reduction of or at the TiO₂ surface and oxidation of the oxidase enzyme. In effect, the design of this BNP allows oxidizing equivalents to be provided to the enzyme, on-demand, photocatalytically, and without the need for oxygen which is normally required as a co-substrate for the enzyme reaction to proceed.

The choice of the specific redox sites within the redox modifier depends on the kinetics and energetics required for the intramolecular oxidation of the enzyme. In general, these sites are chosen and the BNP design otherwise optimized so as to maximize the rate of the electron transfer reaction with the enzyme so that this is not the limiting step in the enzyme catalyzed reaction. Further, the use of redox sites that are fluorescent in only one redox state, or that fluoresce at different wavelengths in the oxidized and reduced states, allows the photocatalytic nanocatalyst to also function as a photocatalytic nanobiosensor (NBS). In this case, the local concentration of enzyme substrate (for example, glucose) can be determined by monitoring the fluorescence of the redox sites on the NBS.

The applications of the photocatalytic nanocatalysts involve biosynthetic reactions. In particular, the photocatalytic nanocatalyst could be used in biosynthetic processes (often times carried out in mini-reactors) to carry out desired enzyme reactions on a continuous or discontinuous basis without the requirement for oxygen. They could have application both for intracellular diagnostic or therapeutic probes and for industrial biosynthetic processes. The NBSs could be used intracellularly to determine and/or change the concentrations of various metabolites (glucose, for example). They could have application for use in cellular assays in which the intracellular concentration of key metabolic indicators is monitored in live cells in order to study basic cellular processes or determine the effects of toxins or potential drug candidates. Such cellular assays could be used for the high throughput screening of drug candidates and for cell based biosensors for toxin detection.

Among the benefits associated with using an MNP based NBS as opposed to molecular sensing species or dyes are the ability to concentrate the molecular detection species on the surface and to modify the MNP surface further to provide additional functionality.

It is known that surface modification/functionalization of semiconductor nanoparticles (e.g., TiO₂, VO₂ and WO₂) allows, upon photoactivation, charge pairs to be separated across the nanoparticle/modifier interface. This aspect of the invention relates to the use of modifiers that were themselves functionalized with single or multiple redox active sites, appropriately arranged along the backbone of the modifier. This would enable charge (+/−) to be transferred/directed via a “hopping” mechanism down the chemical backbone of the modifier toward the exterior of the modified nanoparticle. Furthermore, the judicious choice of the specific redox active sites would allow the control of both the thermodynamics and kinetics of subsequent electron transfer reactions occurring between the redox active (hopping) sites and other chemical functionality either within the backbone of the modifier (intramolecular) or other near-by, chemical species (intermolecular). Additionally, redox active hopping sites could be incorporated that fluoresce in the oxidized and/or reduced state such that the spatial location of the modified nanoparticles could be determined in real time.

This aspect of the invention involves the construction of a BNP as described above in which the biomolecule which is attached to the TiO₂ through a electrochemically reversible redox site is an oxidase enzyme. An oxidase enzyme (for example, glucose oxidase) is attached to the core TiO₂ particle through the redox modifier such that when the redox site is photoexcited, charge separation occurs at the redox site which results in reduction of or at the TiO₂ surface and oxidation of the oxidase enzyme. Alternatively, this process could be initiated via photoactivation of the core TiO₂ particle. Attachment of the enzyme to the redox modifier could be through a co-factor (FAD, for example) that is required for enzyme activity. In effect, the design of this BNP allows oxidizing equivalents to be provided to the enzyme, on-demand, photocatalytically, without the need for oxygen which is normally required as a co-substrate for the enzyme reaction to proceed. Ideally, the design of the BNP would be such that the enzyme attached to the BNP would not be active (that is, it could not use endogenous oxygen as a source of oxidizing equivalents) until the BNP was photoexcited or photoactivated. This would allow the BNP to be totally unreactive and innocuous until it was activated/excited.

The kinetics and energetics associated with the intramolecular oxidation of the enzyme are determined by the choice of the specific redox sites within the redox modifier. The appropriate redox sites could be metal complexes of ruthenium, osmium, or rhenium. In general, these sites are chosen and the BNP design otherwise optimized so as to maximize the rate of the electron transfer reaction with the enzyme so that this is not the limiting step in the enzyme catalyzed reaction. Further, the use of redox sites that are fluorescent in only one redox state or that fluoresce at different wavelengths in the oxidized and reduced state, allows this photocatalytic nanocatalyst to also function as a photocatalytic nanobiosensor (NBS). In this case, the local concentration of enzyme substrate (for example, glucose) can be determined by monitoring the fluorescence of the redox sites on the NBS. Ideally, it should be possible to do this on a discontinuous basis using photoexcitation or photoactivation without significantly effecting the endogenous concentration of the metabolite being monitored.

One example involves a glucose NBS based on glucose oxidase (GOD). FIG. 18 shows a schematic illustration of one molecular design of the glucose NBS (FIG. 18 A) and how it would be expected to function (FIG. 18 B). The voltage values shown are the estimated formal reduction potentials for the indicated reactions.

Various specific designs could be used in which other metal complexes of (Os, Ru and Re) and spacer groups will be inserted in place of those shown in FIG. 18A. For example, Flavin Adenine Dinucleotide (FAD) could be first attached to the construct shown (without GOD) and then Apo-Glucose Oxidase could be reconstituted with the FAD modified BNP.

FIG. 18 B is a generic depiction of how the glucose NBS would function where “M⁺²” and “M⁺³” represent the reduced and oxidized forms of the metal complex. The formal or half wave potential of the metal complex should be at least 0.1 V greater than −0.12 V for the reaction to proceed rapidly and to completion as indicated. The photocatalytic glucose NBS will be a fluorescent-based sensor for dynamically measuring the intracellular glucose concentration on demand. Photoexcitation or photoactivation is indicated by the lightning bolt, and would initiate the process in which the metal complex would be oxidized to the +3 state, which would then oxidize the FADH₂ bound to the Glucose Oxidase (GOD) to FAD. The last step is the oxidation of glucose to gluconolactone.

This entire process can be followed by monitoring the fluorescence of the metal complex redox sites since they are fluorescent in only the reduced form. Either the rate of change in fluorescence or possibly the steady state fluorescence is expected to be dependent on the glucose concentration.

Important criteria for the choice of the redox sites include the energetics (E_{1/2}) and kinetics associated with the electron transfer reaction between the metal complex and the FAD. In this case, both terpyridyl/bipyridyl dicarboxylate metal complexes and synthetic redox amino acids containing bipyridyl metal complexes of ruthenium, osmium and rhenium will be synthesized, as described above, and evaluated. These metal complex modifiers will be attached to TiO₂, as described above, and shown in FIG. 17.

This aspect of the invention will provide nanocatalysts that can be used without contact to provide oxidizing equivalents to an oxidase enzyme on-demand. It will also provide nanobiosensors which can be introduced into living cells and used to dynamically monitor the intracellular concentrations of important metabolites in real time. Preferably, the nanocatalysts and NBSs are only active when they are photoactivated or photoexcited. The NBSs can be tracked intracellularly since they are fluorescent. Because they are nanoparticle based, other functionality can be incorporated into either the nanocatalysts or the NBSs.

While certain representative embodiments and details have been shown for purposes of illustrating the invention, it will be apparent to those skilled in the art that various changes in the compositions and methods disclosed herein may be made without departing from the scope of the invention, which is defined in the appended claims.

What is claimed is:

1. A modified particle comprising:
a semiconductor particle which is photoactivatable; and
a modifier molecule attached to the semiconductor particle, wherein the modifier molecule includes an electrochemically reversible redox active site which is photo excitable.

2. The modified particle of claim 1 wherein the electrochemically reversible redox site is selected from polypyridyl metal complexes.

3. The modified particle of claim 2, wherein the polypyridyl metal complexes contain ruthenium, osmium, or rhenium.

4. The modified particles of claim 1 wherein the semiconductor particles comprise metal oxides or fullerenes.

5. The modified particles of claim 4 wherein the semiconductor particle is the metal oxide selected from SnO₂, WO₃, ZnO, TiO₂, PbO, V₂O₅, Bi₂O₃, Fe₂O₃, CdO, Cu₂O, or CuO.

6. The modified particles of claim 1 wherein there are at least two electrochemically reversible redox active sites.

7. The modified particles of claim 1 wherein the modifier molecule is fluorescent.

8. The modified particle of claim 1 wherein the modifier molecule is directly attached to the semiconductor particle.

9. The modified particle of claim 1 wherein the modifier molecule is attached indirectly to the semiconductor particle using a spacer group.

10. The modified particle of claim 9 wherein the spacer group contains hydroxyl, carboxyl, or keto groups, or groups containing sulfur or phosphorous atoms.

11. The modified particle of claim 9 wherein the spacer group comprises a chemical compound selected from dopa, dopamine, or modified or unmodified diphenols, hydroxylphenyls, siloxanes, thiols, thiol esters, thiol carbamides, phosphonates, or combinations thereof.

12. The modified particle of claim 1 wherein the modified particle is a modified nanoparticle.

13. The modified particle of claim 1 wherein the modified particle is conjugated with a bioactive molecule.

14. The modified particle of claim 14 wherein the bioactive molecule is an enzyme inhibitor.

15. The modified particle of claim 14 wherein the bioactive molecule is an oxidase enzyme.

16. The modified particle of claim 16 wherein the oxidase enzyme is selected from glucose oxidase, amino acid oxidase, lactate oxidase, xanthine oxidase, alcohol oxidase, galactose oxidase, choline oxidase, diamine oxidase, billirubin oxidase, ascorbate oxidase, polyphenol oxidase or combinations thereof.

17. A bioconjugated nanoparticle probe comprising:
a semiconductor nanoparticle which is photoactivatable;
a modifier molecule attached to the semiconductor particle, wherein the modifier molecule includes an electrochemically reversible redox active site which is photoexcitable; and
a bioactive molecule attached to the modifier molecule.

18. The bioconjugate nanoparticle probe of claim 17 wherein the electrochemically reversible redox site is selected from polypyridyl metal complexes.

19. The bioconjugate nanoparticle probe of claim 17 wherein the semiconductor nanoparticle is a metal oxide selected from SnO₂, WO₃, ZnO, TiO₂, PbO, V₂O₅, Bi₂O₃, Fe₂O₃, CdO, Cu₂O, or CuO.

20. The bioconjugate nanoparticle probe of claim 17 wherein the semiconductor nanoparticle is TiO₂, the electrochemically reversible redox site is selected from polypyridyl ruthenium (II) complexes, and the bioactive molecule is an enzyme inhibitor.

21. A method of reversibly inhibiting enzyme activity comprising:
providing a modified particle in a solution comprising:
a semiconductor particle which is photoactivatable;
a modifier molecule attached to the semiconductor particle, wherein the modifier molecule includes an electrochemically reversible redox active site which is photoexcitable; and
an enzyme inhibitor attached to the modifier molecule; and
photoactivating, photoexciting, or both the modified particle so that charge flow is directed in a controlled manner from a core of the modified particle to an exterior surface of the modified particle and wherein the electrochemically reversible redox active site is oxidized or reduced and the exterior surface of the semiconductor particle or a species at the solution/semiconductor particle surface interface is reduced or oxidized and wherein the oxidized or reduced electrochemically reversible redox active site in the modifier molecule subsequently oxidizes or reduces the enzyme inhibitor attached to the modifier molecule rendering the enzyme inhibitor non-inhibiting.

22. A method of monitoring intracellular redox status comprising:
providing a redox nanosensor comprising a modified nanoparticle in a solution comprising:
a semiconductor nanoparticle which is photoactivatable;
a modifier molecule attached to the semiconductor nanoparticle, wherein the modifier molecule includes an electrochemically reversible redox active site which is photoexcitable; and
photoactivating, photoexciting, or both the modified nanoparticle so that charge flow is directed in a controlled manner from a core of the modified nanoparticle to an exterior surface of the modified nanoparticle and wherein the electrochemically reversible redox active site is oxidized or reduced and the exterior surface of the metal oxide particle or a species at the solution/semiconductor particle surface interface is reduced or oxidized; and
monitoring the fluorescence of the redox nanosensor.

23. A method of catalyzing an enzyme reaction comprising:
providing a modified nanoparticle in a solution comprising:
a semiconductor nanoparticle which is photoactivatable;
a modifier molecule attached to the semiconductor nanoparticle, wherein the modifier molecule includes an electrochemically reversible redox active site which is photoexcitable; and
an oxidase enzyme attached to the modifier molecule; and
photoactivating, photoexciting, or both the modified nanoparticle so that charge flow is directed in a controlled manner from a core of the modified particle to an exterior surface of the modified nanoparticle and wherein the electrochemically reversible redox active site is oxidized or reduced and the exterior surface of the metal oxide particle or a species at the solution/semiconductor particle surface interface is reduced or oxidized and wherein the oxidized or reduced electrochemically reversible redox active site in the modifier molecule subsequently oxidizes or reduces the enzyme attached to the modifier molecule in order to catalyze or activate the oxidase enzyme reaction.
